(19) United States
(12) Patent Application Publication
(10) Pub. No.: US 2025/0116674 A1
(43) Pub. Date: Apr. 10, 2025

(54) METHODS FOR PREDICTION AND MONITORING OF SPONTANEOUS PRETERM BIRTH

(71) Applicants: INSERM (INSTITUT NATIONAL DE LA SANTÉ ET DE LA RECHERCHE MÉDICALE), Paris (FR); CHU DE ST ETIENNE, St Etienne (FR); COMMISSARIAT À L'ÉNERGIE ATOMIQUE ET AUX ÉNERGIES ALTERNATIVES (CEA), Paris (FR); UNIVERSITÉ GRENOBLE ALPES, Saint Martin d'Heres (FR); UNIVERSITE JEAN MONNET SAINT ETIENNE, Saint Etienne (FR)

(72) Inventors: Nadia ALFAIDY-BENHAROUGA, Grenoble (FR); Mohamed BENHAROUGA, Grenoble (FR); Céline CHAULEUR, Saint Etienne (FR); Tiphaine BARJAT, Saint Etienne (FR)

(21) Appl. No.: 18/730,454

(22) PCT Filed: Feb. 9, 2023
(86) PCT No.: PCT/EP2023/053158

§ 371 Date: Jul. 19, 2024

(30) Foreign Application Priority Data
Feb. 10, 2022 (EP) 22305143.4

(51) Int. Cl. G01N 33/68

(52) U.S. Cl. CPC G01N 33/689; G01N 33/6872; G01N 2800/368

(57) ABSTRACT

In the present invention, thanks to a prospective multicenter cohort study including 200 pregnant patients with five-serum sampling per patient, inventors investigated the concentrations of protein biomarkers in the plasma of high risk pregnant women during the second and third trimesters to predict spontaneous preterm birth. Inventors demonstrated that PROK1 (Prokineticin 1) also called EG-VEGF (Endocrine Gland-derived Vascular Endothelial Growth Factor), is secreted by the placenta and exhibits increased serum levels in patients with sPTB, before 37 weeks of gestation, compared with uncomplicated pregnant women. More precisely, Women with spontaneous preterm birth exhibited higher concentrations of serum PROK1/EG-VEGF than uncomplicated patients at 20 weeks, 24 weeks, 28 weeks and 32 weeks. Accordingly, serum PROK1/EG-VEGF concentrations could be considered as biomarker of spontaneous preterm birth in high-risk pregnant women.

### FIELD OF THE INVENTION

The present invention relates to methods and kits for prediction and monitoring the spontaneous preterm birth. More specifically, the present invention relates to methods for the prevention of the spontaneous preterm birth through the detection of a specific serum protein biomarker (PROK1/Prokineticin 1) in a patient. This protein is also called EG-VEGF (Endocrine gland derived-Vascular endothelial growth factor).

### BACKGROUND OF THE INVENTION

Preterm birth is defined as all births occurring before 37 weeks of gestation by the Women Health Organization (1). It concerns 10.6% of livebirths worldwide in 2014 (2). Spontaneous preterm birth (sPTB), due to spontaneous onset of labor represents 40 to 45% of preterm births and premature rupture of membranes (PROM) represents 25 to 30% of preterm births (3). To date, sPTB is the first cause of neonatal mortality and morbidity (4,5). The risk factors for sPTB are, a prior preterm birth, black race, periodontal disease, and low maternal body-mass index (3). Prediction of spontaneous preterm birth (sPTB) in asymptomatic women remains a great challenge for the public health system. A short cervical length and a raised cervical-vaginal fetal fibronectin concentration are predictors of spontaneous preterm birth, but their performance remains very low (6). Effective and safety screening tools are still not available in clinical practice, such as the use of amniocentesis-based predictive risk models that are still under investigations (7,8). Early identification of women who will exhibit sPTB will allow intensification of the patient monitoring, tocolytic or vaginal Progesterone medications, pessary and/or cervical Cerclage placement, and decision taking for antenatal corticosteroid therapy.

Prokineticins (PROK) are secreted peptides with a capacity to control both angiogenic and inflammatory processes in humans and in other species (9-11). The PROK family accounts two members, PROK1 and PROK2 (9). The canonical member of this family, PROK1, also known as Endocrine Gland-derived Vascular Endothelial Growth Factor (EG-VEGF). EG-VEGF and PROK2 act through specific G protein-coupled receptors, PROK receptor 1 (PROKR1) and PROK receptor 2 (PROKR2) to control multiple biological functions such as, angiogenesis, circadian rhythm, neurogenesis of olfactory bulb, neuronal survival, reproduction and inflammation (11). EG-VEGF and PROKR1 are highly expressed in first trimester and in term placenta and immunolocalized to different cells types including, syncytiotrophoblasts, cytotrophoblasts, fetal endothelial cells and macrophages (11). Several studies reported the direct involvement of EG-VEGF and its receptors in the etiology of Recurrent Pregnancy Loss (RPL), Gestational Trophoblastic Diseases (GTD) and placenta mediated complications (PMC) (12). These data strongly suggest that the increase in EG-VEGF levels may contribute to the development of PMC or rather participates into the overall compensatory mechanism that occurs to allow the pregnancy to progress.

In relation to chorioamnionitis and parturition, reports from inventor's group and from the group of Jabbour strongly suggested that deregulations in the levels of expression of members of the prokineticin family, including their receptors might be associated with the etiology of this condition (13-16). However, no prospective study involving women at high-risk pregnancies has so far been conducted to determine EG-VEGF levels from early second trimester of pregnancy with the perspective of considering its usefulness as a new biomarker of sPTB.

### SUMMARY OF THE INVENTION

A first object of the present invention relates to an in vitro method for assessing a subject's risk of having or developing spontaneous preterm birth at early stage, comprising the steps of i) determining in a blood sample obtained from the subject the level of the protein PROK1 marker, ii) comparing the level determined in step i) with a reference value and iii) concluding when the level of PROK1 marker determined at step i) is higher than the reference value, is predictive of a high risk of having or developing severe or spontaneous preterm birth.

An additional object of the invention relates to an in vitro method for monitoring subject's risk of having or developing a spontaneous preterm birth comprising the steps of i) determining the level of the protein PROK1 marker in a blood sample obtained from the subject at a first specific time of the disease, ii) determining the level of the protein PROK1 marker in a blood sample obtained from the subject at a second specific time of the disease, iii) comparing the level determined at step i) with the level determined at step ii) and iv) concluding that the risk of having or developing a spontaneous preterm birth has worsen when the level determined at step ii) is higher than the level determined at step i).

An additional object of the invention relates to an in vitro method for monitoring the treatment of a of spontaneous preterm birth comprising the steps of i) determining the level of protein PROK1 marker in a blood sample obtained from the subject before the treatment, ii) determining the level of a of protein PROK1 marker in a blood sample obtained from the subject after the treatment, iii) comparing the level determined at step i) with the level determined at step ii) and iv) concluding that the treatment is efficient when the level determined at step ii) is lower than the level determined at step i).

### DETAILED DESCRIPTION OF THE INVENTION

In the present invention, thanks to a prospective multicenter cohort study including 200 pregnant patients with five-serum sampling per patient, inventors investigated the concentrations of protein biomarkers in the plasma of pregnant women at high risk in the second and third trimesters for the prediction of spontaneous preterm birth. Inventors demonstrated that PROK1 (Prokineticin 1) also called EG-VEGF (Endocrine Gland-derived Vascular Endothelial Growth Factor), is secreted by the placenta and exhibits increased serum levels in patients with sPTB before 37 weeks of gestation, compared with uncomplicated pregnant women. More precisely, Women with spontaneous preterm birth exhibited higher concentrations of serum PROK1/EG-VEGF than uncomplicated patients at 20 weeks, 24 weeks, 28 weeks and 32 weeks. Finally, serum PROK1/EG-VEGF concentrations could be considered as biomarker of spontaneous preterm birth long time before the occurrence of sPTB in asymptomatic pregnant women.

### Prediction Methods According to the Invention

The present invention relates to an in vitro method for assessing a subject's risk of having or developing spontaneous preterm birth, comprising the steps of i) determining in a blood sample obtained from the subject the level of protein PROK1 marker, ii) comparing the level determined in step i) with a reference value and iii) concluding when the level of protein PROK1 marker determined at step i) is higher than the reference value is predictive of a high risk of having or developing spontaneous preterm birth.

In another term, the present invention relates to an in vitro prediction method of having or developing spontaneous preterm birth in a subject, comprising the steps of i) determining in a blood sample obtained from the subject the level of protein PROK1 marker, ii) comparing the level determined in step i) with a reference value and iii) concluding when the level of protein PROK1 marker determined at step i) is higher than the reference value is predictive of having or developing severe or spontaneous preterm birth.

The term “prediction” is a medical term for predicting the likely or expected development of a disease. Predictive scoring is also used for disease outcome predictions.

In the context of the present invention, High PROK1/EG-VEGF levels may constitute, both a prediction marker and a risk factor for developing spontaneous preterm birth.

The term “subject” as used herein refers to a mammalian, such as a rodent (e.g. a mouse or a rat), a feline, a canine or a primate. In a preferred embodiment, said subject is a human subject. The subject according to the invention can be a healthy subject or a subject suffering from a given disease.

In particular embodiments, the subject of the present invention did not suffer from preeclampsia and/or have not been previously diagnosed with preeclampsia.

The term “Preterm birth” is commonly defined as any birth that occur before 37 completed weeks of gestation by the Women Health Organization. It affected 10.6% of livebirths worldwide in 2014.

The “spontaneous preterm birth” (or “sPTB”) is due to spontaneous onset of labor and represents 40 to 45% of all preterm births and “the premature rupture of membranes” (or “PROM”) represent 25 to 30%. To date, sPTB represents the first cause of neonatal mortality and morbidity and prediction sPTB in asymptomatic women remains a great challenge for clinicians. Effective and safety screening tools are still not available in clinical practice for sPTB. However, early identification of women with high risk of sPTB will allow, intensifying the monitoring of their pregnancy through tocolytic or vaginal Progesterone medications, pessary and/or cervical Cerclage placement and, antenatal corticosteroid therapy.

As used herein, the term “sample” or “biological sample” as used herein refers to any biological sample of a subject and can include, by way of example and not limitation, bodily fluids and/or tissue extracts such as homogenates or solubilized tissue obtained from a subject. Tissue extracts are obtained routinely from tissue biopsy. In a particular embodiment regarding the prognostic method of the spontaneous preterm birth according to the invention, the biological sample is a body fluid sample (such blood) or tissue biopsy (placenta) of said subject.

In preferred embodiments, the fluid sample is a blood sample. The term “blood sample” means a whole blood sample obtained from a subject (e.g. an individual for which it is interesting to determine whether a population of biomarkers can be identified).

As used herein, the term “prokineticin 1” or “PROK1”, also known as “Endocrine Gland-derived Vascular Endothelial Growth Factor” (EG-VEGF)” is a protein that in humans is encoded by the OLR1 gene. (human gene: Gene ID: 84432/Entrez Gene: PROK1 prokineticin 1: www.ncbi.nlm.nih.gov/gene=84432). Prokineticins (PROK) are secreted peptides with a capacity to control both angiogenic and inflammatory processes in humans and in other species (9-11). PROK1 and PROK2 are the 2 key members of the Prokineticins (PROK) family (9). PROK1 (EG-VEGF) and PROK2 act through specific G protein-coupled receptors, PROK receptor 1 (PROKR1) and PROK receptor 2 (PROKR2) to control multiple biological functions such as, angiogenesis, circadian rhythm, neurogenesis of olfactory bulb, neuronal survival, reproduction, and inflammation (11). PROK1 (EG-VEGF) and PROKR1 are highly expressed in first trimester and in term placenta and immunolocalized to different cells types including, syncytiotrophoblasts, cytotrophoblasts, fetal endothelial cells and macrophages (11). Several studies reported the direct involvement of EG-VEGF and its receptors in the etiology of Recurrent Pregnancy Loss (RPL), Gestational Trophoblastic Diseases (GTD) (PMC) (12, 24, 25, 26, 27). These data strongly suggest that the increase in EG-VEGF levels may contribute to the development of PMC or rather participates into the overall compensatory mechanism that occur to allow the pregnancy to progress One example of PROK1 human amino acid sequence (UniProtKB-P58294) is provided in NCBI database: NCBI Reference Sequence: NP_115790 (prokineticin-1 precursor).

One example of nucleotide sequence encoding wild-type huma PROK1 is provided in in NCBI database: NCBI Reference Sequence: NM_032414 (prokineticin-1 precursor)).

Of course variant sequences of the PROK1 may be used in the context of the present invention (as biomarker), those including but not limited to functional homologues, paralogues or orthologues, transcript variants of such sequences

The level of the PROK1 may be determined by using standard electrophoretic and immunodiagnostic techniques, including immunoassays such as competition, direct reaction such as immunohistochemistry, or sandwich type assays. Such assays include, but are not limited to, Western blots; agglutination tests; enzyme-labelled and mediated immunoassays, such as ELISAs; biotin/avidin type assays; radioimmunoassays; immunoelectrophoresis; immunoprecipitation, etc. The reactions generally include revealing labels such as fluorescent, chemiluminescent, radioactive, enzymatic labels or dye molecules, or other methods for detecting the formation of a complex between the antigen and the antibody or antibodies reacted therewith.

For example, determination of the PROK1 level can be performed by a variety of techniques and method any well-known method in the art: ELISA kits (PeproTech, France, Invitrogen™ Kit ELISA humain EG-VEGF/PROK1) RIA kits Immunochemiluminescent methods.

In a particular embodiment, the methods of the invention comprise contacting the blood sample with a binding partner.

As used therein, binding partner refers to a molecule capable of selectively interacting with PROK1.

The binding partner may be generally an antibody that may be polyclonal or monoclonal, preferably monoclonal. Polyclonal antibodies directed against PROK1 can be raised according to known methods by administering the appropriate antigen or epitope to a host animal selected, e.g., from pigs, cows, horses, rabbits, goats, sheep, and mice, among others. Various adjuvants known in the art can be used to enhance antibody production. Although antibodies useful in practicing the invention can be polyclonal, monoclonal antibodies are preferred. Monoclonal antibodies against PROK1 can be prepared and isolated using any technique that provides for the production of antibody molecules by continuous cell lines in culture. Techniques for production and isolation include are disclosed above. Antibodies useful in practicing the present invention also include anti-PROK1 including but not limited to F(ab′)2 fragments, which can be generated by pepsin digestion of an intact antibody molecule, and Fab fragments, which can be generated by reducing the disulfide bridges of the F(ab′)2 fragments. Alternatively, Fab and/or scFv expression libraries can be constructed to allow rapid identification of fragments having the desired specificity to PROK1. For example, phage display of antibodies may be used. In such a method, single-chain Fv (scFv) or Fab fragments are expressed on the surface of a suitable bacteriophage, e.g., M13. Briefly, spleen cells of a suitable host, e.g., mouse that has been immunized with a protein are removed. The coding regions of the VL and VH chains are obtained from those cells that are producing the desired antibody against the protein. These coding regions are then fused to a terminus of a phage sequence. Once the phage is inserted into a suitable carrier, e. g., bacteria, the phage displays the antibody fragment. Phage display of antibodies may also be provided by combinatorial methods known to those skilled in the art. Antibody fragments displayed by a phage may then be used as part of an immunoassay.

In another embodiment, the binding partner may be an aptamer as described above.

The binding partners of the invention such as antibodies or aptamers, may be labeled with a detectable molecule or substance, such as a fluorescent molecule, a radioactive molecule or any others labels known in the art. Labels are known in the art that generally provide (either directly or indirectly) a signal.

As used herein, the term “labeled”, with regard to the binding partner, is intended to encompass direct labeling of the antibody or aptamer by coupling (i.e., physically linking) a detectable substance, such as a radioactive agent or a fluorophore (e.g. fluorescein isothiocyanate (FITC) or phycoerythrin (PE) or Indocyanine (Cy5)) to the antibody or aptamer, as well as indirect labeling of the probe or antibody by reactivity with a detectable substance. An antibody or aptamer of the invention may be labeled with a radioactive molecule by any method known in the art. For example, radioactive molecules include but are not limited radioactive atom for scintigraphic studies such as 1123, 1124, In111, Re186, Re188.

The aforementioned assays generally involve the bounding of the binding partner (i.e. antibody or aptamer) in a solid support. Solid supports which can be used in the practice of the invention include substrates such as nitrocellulose (e. g., in membrane or microtiter well form); polyvinylchloride (e. g., sheets or microtiter wells); polystyrene latex (e.g., beads or microtiter plates); polyvinylidine fluoride; diazotized paper; nylon membranes; activated beads, magnetically responsive beads, and the like. More particularly, an ELISA method can be used, wherein the wells of a microtiter plate are coated with a set of antibodies against PROK1 protein. A blood sample containing or suspected of containing PROK1 is then added to the coated wells. After a period of incubation sufficient to allow the formation of binding partner-PROK1 complexes, the plate(s) can be washed to remove unbound material and a labeled secondary binding molecule added. The secondary binding molecule is allowed to react with any captured sample marker protein, the plate washed and the presence of the secondary binding molecule detected using methods well known in the art.

As the binding partner, the secondary binding molecule may be labeled.

Different immunoassays, such as radioimmunoassay or ELISA, have been described in the art.

Measuring the level of PROK1 protein with or without immunoassay-based methods may also include separation of the proteins: centrifugation based on the protein's molecular weight; electrophoresis based on mass and charge; HPLC based on hydrophobicity; size exclusion chromatography based on size; and solid-phase affinity based on the protein's affinity for the particular solid-phase that is used. Once separated, PROK1 may be identified based on the known “separation profile” e. g., retention time, for that protein and measured using standard techniques. Alternatively, the separated proteins may be detected and measured by, for example, a mass spectrometer.

In a preferred embodiment, the method for measuring the level of PROK1 comprises the step of contacting the blood sample with a binding partner capable of selectively interacting with PROK1 to allow formation of a binding partner-PROK1 complex.

In more preferred embodiment, the method according to the invention comprises further the steps of separating any unbound material of the blood sample from the binding partner-PROK1 complex, contacting the binding partner-PROK1 complex with a labelled secondary binding molecule, separating any unbound secondary binding molecule from secondary binding molecule-PROK1 complexes and measuring the level of the secondary binding molecule of the secondary binding molecule-PROK1 complexes.

Typically, a high or a low level of PROK1 is intended by comparison to a control reference value.

Accordingly, in a particular embodiment, the prognostic method of the present invention comprising the step of comparing said level of PROK1 to a control reference value wherein
A high level of PROK1 marker is predictive of a high risk of having or developing a spontaneous preterm birth.
A low level of PROK1 marker is predictive of a low risk of having or developing a spontaneous preterm birth.

Said reference control values may be determined in regard to the level of PROK1 present in blood sample taken from one or more healthy subject or to the PROK1 distribution in a control population.

In one embodiment, the method according to the present invention comprises the step of comparing said level of PROK1 to a control reference value, wherein a high level of PROK1 marker compared to said control reference value is predictive of a high risk of having a spontaneous preterm birth and a low level of PROK1 marker compared to said control reference value is predictive of a low risk of having a spontaneous preterm birth.

The control reference value may depend on various parameters such as the method used to measure the level of PROK1 of the subject.

Typically, regarding the reference value of “PROK1 marker” at 32 week of gestation, as indicated in the Example section (FIG. 2 and Table 2), using Elisa technology for marker dosage; a serum level of EG-VEGF superior to 195 μg/ml is predictive of having or a high risk of having or developing a spontaneous preterm birth and a serum level of EG-VEGF lower than 195 μg/ml is predictive of, not having, developing or being at a low risk of having a spontaneous preterm birth.

Control reference values are easily determinable by the one skilled in the art, by using the same techniques as for determining the level of PROK1 in blood sample previously collected from the patient under testing.

A “reference value” can be a “threshold value” or a “cut-off value”. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data (see FIG. 2). Preferably, the person skilled in the art may compare the level of PROK1 (protein or nucleic sequence (mRNA)) of the present invention with a defined threshold value. In one embodiment of the present invention, the threshold value is derived from the PROK1 protein level (or ratio, or score) determined in a blood sample derived from one or more subjects who are responders (to the method according to the invention). In one embodiment of the present invention, the threshold value may also be derived from PROK1 protein level (or ratio, or score) determined in a blood sample derived from one or more subjects or who are non-responders. Furthermore, retrospective measurement of the PROK1 protein level (or ratio, or scores) in properly banked historical subject samples may be used in establishing these threshold values.

For example, after determining the expression level of the PROK1 protein expression (protein or nucleic sequence (mRNA)) in a group of reference, one can use algorithmic analysis for the statistic treatment of the expression levels determined in blood samples to be tested, and thus obtain a classification standard having significance for blood sample classification. The full name of ROC curve is receiver operator characteristic curve, which is also known as receiver operation characteristic curve. It is mainly used for clinical biochemical diagnostic tests. ROC curve is a comprehensive indicator that reflects the continuous variables of true positive rate (sensitivity) and false positive rate (1-specificity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Then sensitivity is used as the vertical coordinate and specificity is used as the horizontal coordinate to draw a curve. The higher the area under the curve (AUC), the higher the accuracy of diagnosis. On the ROC curve, the point closest to the far upper left of the coordinate diagram is a critical point having both high sensitivity and high specificity values. The AUC value of the ROC curve is between 1.0 and 0.5. When AUC>0.5, the diagnostic result gets better and better as AUC approaches 1. When AUC is between 0.5 and 0.7, the accuracy is low. When AUC is between 0.7 and 0.9, the accuracy is moderate. When AUC is higher than 0.9, the accuracy is high. This algorithmic method is preferably performed by a computer. Existing software or systems in the art may be used for the drawing of the ROC curve, such as: MedCalc 9.2.0.1 medical statistical software, SPSS 9.0, ROCPOWER.SAS, DESIGNROC.FOR, MULTIREADER POWER.SAS, CREATE-ROC.SAS, GB STAT VI0.0 (Dynamic Microsystems, Inc. Silver Spring, Md., USA), etc.

In some embodiments, the method of the invention comprises the use of a classification algorithm typically selected from Linear Discriminant Analysis (LDA), Topological Data Analysis (TDA), Neural Networks, Support Vector Machine (SVM) algorithm and Random Forests algorithm (RF). In some embodiments, the method of the invention comprises the step of determining the subject response using a classification algorithm. As used herein, the term “classification algorithm” has its general meaning in the art and refers to classification and regression tree methods and multivariate classification well known in the art such as described in U.S. Pat. No. 8,126,690; WO2008/156617. As used herein, the term “support vector machine (SVM)” is a universal learning machine useful for pattern recognition, whose decision surface is parameterized by a set of support vectors and a set of corresponding weights, refers to a method of not separately processing, but simultaneously processing a plurality of variables. Thus, the support vector machine is useful as a statistical tool for classification. The support vector machine non-linearly maps its n-dimensional input space into a high dimensional feature space, and presents an optimal interface (optimal parting plane) between features. The support vector machine comprises two phases: a training phase and a testing phase. In the training phase, support vectors are produced, while estimation is performed according to a specific rule in the testing phase. In general, SVMs provide a model for use in classifying each of n subjects to two or more disease categories based on one k-dimensional vector (called a k-tuple) of biomarker measurements per subject. An SVM first transforms the k-tuples using a kernel function into a space of equal or higher dimension. The kernel function projects the data into a space where the categories can be better separated using hyperplanes than would be possible in the original data space. To determine the hyperplanes with which to discriminate between categories, a set of support vectors, which lie closest to the boundary between the disease categories, may be chosen. A hyperplane is then selected by known SVM techniques such that the distance between the support vectors and the hyperplane is maximal within the bounds of a cost function that penalizes incorrect predictions. This hyperplane is the one which optimally separates the data in terms of prediction (Vapnik, 1998 Statistical Learning Theory. New York: Wiley). Any new observation is then classified as belonging to any one of the categories of interest, based where the observation lies in relation to the hyperplane. When more than two categories are considered, the process is carried out pairwise for all of the categories and those results combined to create a rule to discriminate between all the categories. As used herein, the term “Random Forests algorithm” or “RF” has its general meaning in the art and refers to classification algorithm such as described in U.S. Pat. No. 8,126,690; WO2008/156617. Random Forest is a decision-tree-based classifier that is constructed using an algorithm originally developed by Leo Breiman (Breiman L, “Random forests,” Machine Learning 2001, 45:5-32). The classifier uses a large number of individual decision trees and decides the class by choosing the mode of the classes as determined by the individual trees. The individual trees are constructed using the following algorithm: (1) Assume that the number of cases in the training set is N, and that the number of variables in the classifier is M; (2) Select the number of input variables that will be used to determine the decision at a node of the tree; this number, m should be much less than M; (3) Choose a training set by choosing N samples from the training set with replacement; (4) For each node of the tree randomly select m of the M variables on which to base the decision at that node; (5) Calculate the best split based on these m variables in the training set. In some embodiments, the score is generated by a computer program.

In some embodiments, the method of the present invention comprises a) quantifying the level of PROK1 expression (protein or nucleic sequence (mRNA)) in the blood sample; b) implementing a classification algorithm on data comprising the quantified PROK1 protein so as to obtain an algorithm output; c) determining the probability that the subject will develop a spontaneous preterm birth from the algorithm output of step b).

The algorithm used with the method of the present invention can be performed by one or more programmable processors executing one or more computer programs to perform functions by operating on input data and generating output. The algorithm can also be performed by, and apparatus can also be implemented as, special purpose logic circuitry, e.g., an FPGA (field programmable gate array) or an ASIC (application-specific integrated circuit). Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Moreover, a computer can be embedded in another device. Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry. To provide for interaction with a user, embodiments of the invention can be implemented on a computer having a display device, e.g., in non-limiting examples, a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. Accordingly, in some embodiments, the algorithm can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the invention, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), e.g., the Internet. The computing system can include clients and servers. A client and server are generally remote from each other and typically interact through a communication network. The relationship of client and server arises by virtue of computer programs running on the respective computers and having a client-server relationship to each other. “Risk” in the context of the present invention, relates to the probability that an event will occur over a specific time period, as in the conversion to spontaneous preterm birth, and can mean a subject's “absolute” risk or “relative” risk. Absolute risk can be measured with reference to either actual observation post-measurement for the relevant time cohort, or with reference to index values developed from statistically valid historical cohorts that have been followed for the relevant time period. Relative risk refers to the ratio of absolute risks of a subject compared either to the absolute risks of low risk cohorts or an average population risk, which can vary by how clinical risk factors are assessed. Odds ratios, the proportion of positive events to negative events for a given test result, are also commonly used (odds are calculated according to the formula p/(1-p) where p is the probability of event and (1-p) is the probability of no event) to no conversion. Alternative continuous measures, which may be assessed in the context of the present invention, include time to spontaneous preterm birth, conversion risk reduction ratios.

“Risk evaluation,” or “evaluation of risk” in the context of the present invention encompasses making a prediction of the probability, odds, or likelihood that an event or disease state may occur, the rate of occurrence of the event or conversion from one disease state to another, i.e., from a normal condition to a spontaneous preterm birth condition or to one at risk of developing a spontaneous preterm birth. Risk evaluation can also comprise prediction of future clinical parameters, traditional laboratory risk factor values, or other indices of spontaneous preterm birth, such as cellular population determination in peripheral tissues, in serum or other fluid, either in absolute or relative terms in reference to a previously measured population. The methods of the present invention may be used to make continuous or categorical measurements of the risk of conversion to spontaneous preterm birth, thus prognosing and defining the risk spectrum of a category of subjects defined as being at risk for a spontaneous preterm birth. In the categorical scenario, the invention can be used to discriminate between normal and other subject cohorts at higher risk for spontaneous preterm birth. In other embodiments, the present invention may be used so as to help to discriminate those having spontaneous preterm birth from normal ones.

The invention also relates to the use of PROK1 in a blood sample as a prediction biomarker of spontaneous preterm birth, especially at different stages of the pregnancy. According the present invention, the term “different stages” refers to the stage of the pregnancy at least 20, 21, 22, 23, 24; 25, 26; 27, 28, 29, 30, 31, 32 33, 34; 35, 36; 37 weeks of pregnancy.

Accordingly, the method of detection of the invention is consequently useful for the in vitro prediction of spontaneous preterm birth from a blood sample. In particular, the method of detection of the invention is consequently useful for the in vitro prediction of spontaneous preterm birth at early stage (at least 20 weeks of pregnancy) from a blood sample.

### Monitoring Methods and Management

An additional object of the invention relates to an in vitro method for monitoring a spontaneous preterm birth comprising the steps of i) determining the level of protein PROK1 marker in a blood sample obtained from the subject at a first specific time of the disease, ii) determining the level of protein PROK1 marker in a blood sample obtained from the subject at a second specific time of the disease, iii) comparing the level determined at step i) with the level determined at step ii) and iv) concluding that the risk of having or developing a spontaneous preterm birth has evolved in worse manner when the level determined at step ii) is higher than the level determined at step i).

An additional object of the invention relates to an in vitro method for monitoring the treatment of a spontaneous preterm birth comprising the steps of i) determining the level of protein PROK1 marker in a blood sample obtained from the subject before the treatment, ii) determining the level of protein PROK1 marker in a blood sample obtained from the subject after the treatment”, iii) comparing the level determined at step i) with the level determined at step ii) and iv) concluding that the treatment is efficient when the level determined at step ii) is lower than the level determined at step i).

In particular embodiment, the spontaneous preterm birth is detected at early stage (at least 20 week of pregnancy)

The decrease can be e.g. at least 5%, or at least 10%, or at least 20%, more preferably, at least 50% even more preferably at least 100%.

### Therapeutic Method of a Specific Population

The invention also relates to a method for treating spontaneous preterm birth with an antenatal tocolytic or vaginal Progesterone medications, pessary and/or cervical Cerclage placement and, in a subject wherein the level of protein PROK1 marker obtained from said patient have been detected by one of the methods of the invention.

In the context of the invention, the term “treating” or “treatment”, as used herein, means reversing, alleviating, inhibiting the progress of, or preventing the disorder or condition to which such term applies, or reversing, alleviating, inhibiting the progress of, or preventing one or more symptoms of the disorder or condition to which such term applies.

Another object of the present invention is a method of treating/preventing spontaneous preterm birth in a subject comprising the steps of:
a) providing a blood sample containing neutrophil from a subject,
b) detecting the level of protein PROK1 marker
c) comparing the level determined at stet b) with a reference value and
if level determined at stet b) is higher than the reference value, treating the subject with antenatal Progesterone and/or tocolytic therapy.

As mentioned, antenatal tocolytic or vaginal Progesterone medications, pessary and/or cervical Cerclage placement are the current main treatment for spontaneous preterm birth.

Accordingly, the invention also relates to a method for treating spontaneous preterm birth with antenatal tocolytic or vaginal Progesterone medications, pessary and/or cervical Cerclage placement in a subject wherein the level of protein PROK1 marker obtained from said patient, have been detected by one of the methods of the invention.

Method of treating spontaneous preterm birth in a subject comprising the steps of:
a) providing a blood sample containing neutrophils from a subject,
b) detecting the level of protein PROK1 marker
c) comparing the level determined at stet b) with a reference value and
if level determined at step b) is higher than the reference value, treating the subject with tocolytic or vaginal Progesterone medications, pessary and/or cervical Cerclage placement.

In particular embodiments, the spontaneous preterm is detected at early stage (at least 20 week of pregnancy

The term “progesterone” means a medication and naturally occurring steroid hormone. Progesterone therapy is a “progestogen” (also referred to as a progestagen, gestagen, or gestogen, is a type of medication which produces effects similar to progesterone) and is used in combination with estrogens mainly in hormone therapy for menopausal symptoms and low sex hormone levels in women. It is also used in women to support pregnancy and fertility and to treat gynaecological disorders. Progesterone can be taken by mouth, through the vagina, and by injection into muscle or fat, among other routes. A large number of synthetic progestogens, or progestins, have been derived from progesterone and are used as medications as well (Kuhl H (2005). Climacteric. 8 Suppl 1: 3-63). Examples include medroxyprogesterone acetate and norethisterone.

The term “Tocolytics” also called “anti-contraction medications” or “labor suppressants” are medications used to suppress premature labour. Therefore, tocolytic therapy is provided when delivery would result in premature birth, postponing delivery long enough for the administration of glucocorticoids, which accelerate fetal lung maturity but may require one to two days to take effect.

Commonly used tocolytic medications include, Oxytocin receptor antagonist (such as Atosiban) β2 agonists (see below), calcium channel blockers (such as nifedipine (Procardia, Adalat)) a, NSAIDs (such as Indomethacin or Sulindac) and magnesium sulfate. These can assist in delaying preterm delivery by suppressing uterine muscle contractions and their use is intended to reduce fetal morbidity and mortality associated with preterm birth (Mayer C. et al (2021), “Tocolysis”, StatPearls, Treasure Island (FL): StatPearls Publishing) The suppression of contractions is often only partial and tocolytics can only be relied on to delay birth for a matter of days. Depending on the tocolytic used, the pregnant woman or fetus may require monitoring (e.g., blood pressure monitoring when nifedipine is used as it reduces blood pressure; cardiotocography to assess fetal well-being.

Example of J2 agonists used as tocolytics can be selected from the list consisting of, Salbutamol (INN) or albuterol (USAN), Fenoterol, Terbutaline (Brethine), Ritodrine (Yutopar), Hexoprenaline (Gynipral).

In particular embodiment the tocolytic drug is nifedipine one of the most commonly used tocolytic agents.

The invention will be further illustrated by the following figures and examples. However, these examples and figures should not be interpreted in any way as limiting the scope of the present invention.

### FIGURES

FIG. 1: Circulating PROK1/EG-VEGF concentrations at four gestational ages in uncomplicated and spontaneous preterm birth pregnant women. The central horizontal bars are the medians. The lower and upper limits of the boxes are the first and third quartiles. * p<0.05**<0.001.

FIG. 2: ROC curves analysis of Circulating PROK1/EG-VEGF concentrations for predicting spontaneous preterm birth at 32 weeks. AUC: area under the curve

### EXAMPLE

### Methods:

### Study Design and Population

Our study is based on data from the AngioPred study as previously described (17). The AngioPred study is a prospective multicenter cohort study conducted between June 2008 and October 2010 in the Obstetrics and Gynecology department of Saint Etienne and Nimes University Hospitals, and the Laboratory of Hematology in Nimes University Hospital. The patients included in this study had consulted within 20 weeks, and were all at high risk for occurrence or recurrence of (PMC).

The patients included were all at high risk for occurrence or recurrence of PMCs. The risks included, diabetes, chronic hypertension, obesity, maternal age younger than 18 years or older than 38 years, chronic kidney disease, systemic lupus erythematosus, antiphospholipid syndrome, family history of cardiovascular disease or venous thromboembolism in first degree relatives, biological thrombophilia without any personal history of venous thromboembolism or of PMC, a history of one or more episodes of PMCs or personal history of venous thromboembolism. The exclusion criteria were the following, twin pregnancies, patients with a history of fetal death, IUGR which etiology was of chromosomal, genetic or infectious origin, and the presence of any PMC or venous thromboembolism at inclusion.

The Ethics Committee and Institutional Review Board of the University Hospital of Saint Etienne approved the protocol in March 2008. The study was registered on clinicaltrials.gov (identifier NCT00695942). The clinical investigation was performed according to the Helsinki Declaration of 1975, as revised in 1996. All patients were included before 20 weeks of gestation and gave their written consent.

### Outcomes

The primary outcome was the occurrence of spontaneous preterm birth, defined as the number of spontaneous commencement of labor with intact or pre-labor rupture of membranes and birth at or after 20 weeks and 0 days of gestation, and before 37 weeks and 0 days of gestation.

### Blood Collection

Blood samples were collected at the collection center of the University Hospital of Saint-Etienne and Nimes at 20, 24, 28, 32, and 36 weeks of gestation, totaling five samples per patient. The samples were immediately sent to laboratories for analysis. The samples were centrifuged, aliquoted, and stored at −80° C.

### Biological Analysis

Each analysis was performed in blind manner to other analyses. All samples from the same patient were grouped in the same series of assays. Analyses were carried out after defrosting for 10 min in a water bath at 37° C. and centrifugation at 2500 g. Serum EG-VEGF levels were measured at 20, 24, 28, 32, and 36 weeks by an enzyme-linked immunosorbent assay (ELISA) kit (PeproTech, Neuilly-Sur-Seine, France) with a standard range between 16-1000 μg/mL. Two separate standard curves were constructed to allow accurate readings of samples at the upper and lower ranges of the assays.

### Statistical Analysis

Statistical analyses were performed using XISTAT®. Qualitative data were presented as absolute and relative frequencies (expressed in %). The qualitative variables were compared by the Chi-square test or by Fisher's exact test if the numbers were insufficient. Quantitative variables were presented as mean and standard deviation. Normal distribution of data was tested with the Shapiro-Wilk test. Results were reported as boxplots. The threshold value of EG-VEGF plasma levels for the prediction of spontaneous preterm birth was determined at each gestational age through the receiver operator characteristics (ROC) curve, calculating the area under the curve with 95% confidence intervals (95% CI) (18). All hypothesis tests were performed at the 0.05 significance level, so p<0.05 was considered significant.

### Results

### Clinical Characteristics

Between June 2008 and October 2010, 200 consecutive pregnant women were included in the study. Demographics and inclusion criteria are summarized in Table 1.

During the study, 45 women had a PMC and were excluded from the analysis. Seven women presented a spontaneous preterm birth. All demographic characteristics and inclusion criteria were similar between uncomplicated women and women with sPTB.

Categorical variables reported as frequency (percentage) and continuous variables reported as mean (standard deviation). Abbreviations: BMI: body mass index, MAP: Mean arterial pressure, Mean UARI: mean uterine artery resistance index, VTE: venous thromboembolism, PMC: Placenta-mediated complication

### Serum EG-VEGF Levels and the Occurrence of Spontaneous Preterm Birth

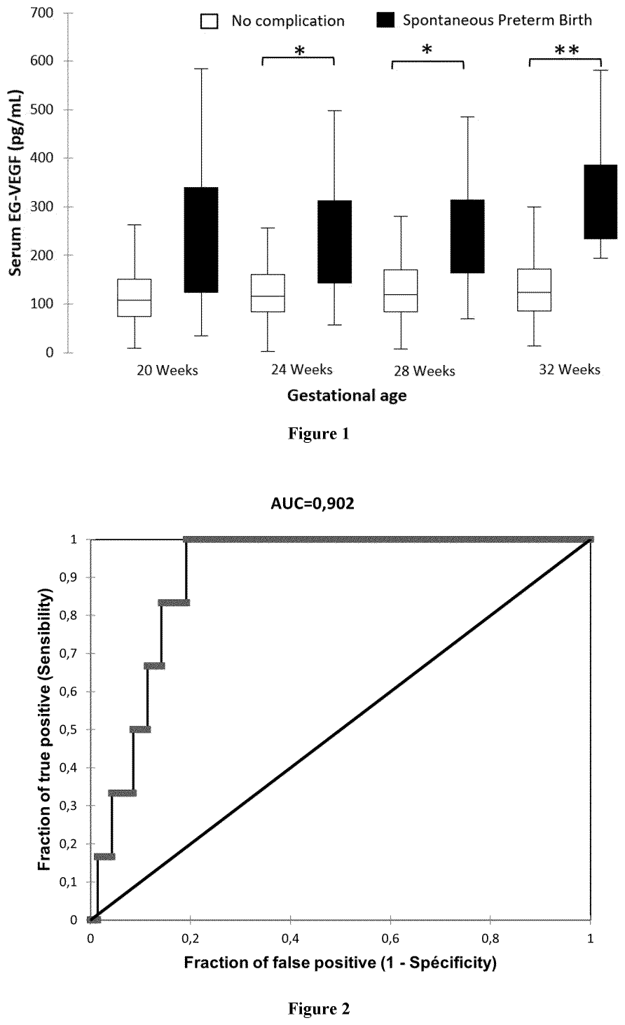

Women with sPTB had higher concentrations of EG-VEGF than uncomplicated patients at 24 weeks (244.1 versus 144.3 pg/mL), 28 weeks (247.5 versus 146.2 pg/mL) and 32 weeks (328.5 versus 152.7 pg/mL) (p=0.03, 0.02 and <0.001). Results are summarized in FIG. 1.

ROC curves were used to calculate the threshold values of serum EG-VEGF levels to exhibit the best sensitivity and specificity for the prediction of sPTB. The areas under the curve (AUC) reached 0.9 with 100% of sensibility at 32 weeks for prediction of spontaneous preterm birth (FIG. 2).

### DISCUSSION

Serum EG-VEGF levels were predictive of spontaneous preterm birth as these levels increased early on, from 24 weeks of gestation. Circulating EG-VEGF exhibited higher levels in women with spontaneous preterm birth with a good prediction capacity at 32 weeks.

Previous reports from our group demonstrated that in normal pregnancy, circulating EG-VEGF levels increased during the third trimester compared to the second trimester, but decreased during labor compared to patients with no labor (19). EG-VEGF is also expressed in the mouse fetal membrane (FM) by the end of gestation, suggesting a local role for this protein in the mechanism of parturition (19). These data suggested to us that EG-VEGF is a cytokine that may act locally to ensure fetal membranes protection in late pregnancy. Hence, EG-VEGF decreased-expression may contribute to the initiation of human labor process, revealed by the abrupt decrease in its levels of expression as well those of its receptors.

So far, no study has assessed the prediction capacity of EG-VEGF level for spontaneous preterm birth. The increase in EG-VEGF levels in sPTB substantiated our assumption regarding the direct involvement of this factor in the etiology of pregnancy-associated pathologies. A compensatory role in this pathology is very likely. Hence, one can speculate that EG-VEGF levels are elevated, not only to reactivate angiogenesis but also to control labor-associated inflammation. Microbiological studies suggest that intrauterine infection may account for 25 to 40% of preterm births (20). Importantly, Jabbour et al (15) tested the potential involvement of EG-VEGF in preterm induction upon its injection in animal models. EG-VEGF was compared to lipopolysaccharide (LPS), a component of the cell wall of the Gram-negative bacteria E. coli, known to induce labor. LPS but not EG-VEGF injection induced preterm delivery within the following 20 h. EG-VEGF injection induced an increase in the mRNA expression of the pro-inflammatory mediators IL-6, IL-1, tumor necrosis factor (TNF), CXCL2, and CXCL5 within the cells of the fetal membranes (21). EG-VEGF also increased the same inflammatory mediators in the human myometrium (22). In addition, monocytes treated with EG-VEGF for 24 h released the chemokines CXCL1, CXCL8, and CCL4, and co-stimulation by LPS lead to a synergic effects on the production of CCL18 and CCL20 (23). While these findings strongly suggest that EG-VEGF is involved in the process that accompany preterm birth, to date we do not know whether its increased concentrations are cause or consequence. Further in vivo studies are ongoing to decipher the significance of EG-VEGF elevation in this pathology.

Altogether, our study demonstrates for the first time that EG-VEGF levels can constitute strong biomarker of the occurrence of sPTB. The strengths of this study are the examination of a population of patients at high risk of PMC who were recruited prospectively and followed from 20 weeks to delivery.

The association of EG-VEGF levels with spontaneous preterm birth will be investigated in larger cohorts to validate its informational value and propose it use in routine assessment of patient with high risk of this pathology.

TABLE 1
Patient characteristics at inclusion

| | | |
|---|---|---|
| | Spontaneous | |
| No complication | Preterm birth | |
| N = 148 | N = 7 | P value |

Maternal characteristics

| | | | |
|---|---|---|---|
| Age, years (SD) | 31.5 (4.9) | 28.3 (6.5) | 0.95 |
| Parity, mean (SD) | 1.2 (1.0) | 1.4 (1.0) | 0.51 |
| BMI (Kg/m²) | 25.0 (5.9) | 29.0 (12.0) | 0.75 |
| Smoking | 17 (11.5) | 2 (28.6) | 0.20 |

Maternal history

| | | | |
|---|---|---|---|
| Diabetes | 5 (3.4) | 0 (0) | 0.62 |
| Kidney disease | 3 (2.0) | 0 (0) | 0.70 |
| Chronic hypertension | 8 (5.4) | 0 (0) | 0.52 |
| Lupus | 11 (7.4) | 2 (28.6) | 0.06 |
| Antiphospholipid syndrome | 5 (3.4) | 0 (0) | 0.62 |
| Personal history of VTE | 28 (18.9) | 3 (42.9) | 0.13 |
| Personal history of PMC | 89 (60.1) | 4 (57.1) | 0.01 |
| Family history of VTE or | 32 (21.6) | 1 (14.3) | 0.62 |
| cardiovascular disease | | | |

Categorical variables reported as frequency (percentage) and continuous variables reported as mean (standard deviation). Abbreviations: BMI: body mass index, MAP: Mean arterial pressure, Mean UARI: mean uterine artery resistance index, VTE: venous thromboembolism, PMC: Placenta-mediated complication.

TABLE 2
Performance of EG-VEGF for prediction
of spontaneous preterm birth

| EG-VGEF | | | | | | | |
|---|---|---|---|---|---|---|---|
| cutoff | Gestational | | | | | | |
| (pg/mL) | age | AUC | p value | Se | Sp | PPV | NPV |
| 155.32 | 20 weeks | 0.698 | 0.15 | 71.4 | 77.0 | 12.8 | 98.3 |
| 242.34 | 24 weeks | 0.741 | 0.04 | 57.1 | 89.4 | 21.1 | 97.7 |
| 197.14 | 28 weeks | 0.762 | 0.017 | 71.4 | 84.3 | 18.5 | 98.3 |
| 194.84 | 32 weeks | 0.902 | <0.001 | 100 | 80.9 | 18.2 | 100 |

AUC: area under curve,
Se: sensibility,
Sp: specificity,
PPV: predictive positive value,
NPV: negative predictive value

### REFERENCES

Throughout this application, various references describe the state of the art to which this invention pertains. The disclosures of these references are hereby incorporated by reference into the present disclosure.
1. WHO: recommended definitions, terminology and format for statistical tables related to the perinatal period and use of a new certificate for cause of perinatal deaths. Modifications recommended by FIGO as amended Oct. 14, 1976. Acta Obstet Gynecol Scand. 1977; 56(3):247-53.
2. Chawanpaiboon S, Vogel J P, Moller A-B, Lumbiganon P, Petzold M, Hogan D, et al. Global, regional, and national estimates of levels of preterm birth in 2014: a systematic review and modelling analysis. Lancet Glob Health. janv 2019; 7(1):e37-46.
3. Goldenberg R L, Culhane J F, Jams J D, Romero R. Epidemiology and causes of preterm birth. Lancet. 5 janv 2008; 371(9606):75-84.
4. Patel R M. Short- and Long-Term Outcomes for Extremely Preterm Infants. Am J Perinatol. fevr 2016; 33(3):318-28.
5. Natarajan G, Shankaran S. Short- and Long-Term Outcomes of Moderate and Late Preterm Infants. Am J Perinatol. fevr 2016; 33(3):305-17.
6. Esplin M S, Elovitz M A, Jams J D, Parker C B, Wapner R J, Grobman W A, et al. Predictive Accuracy of Serial Transvaginal Cervical Lengths and Quantitative Vaginal Fetal Fibronectin Levels for Spontaneous Preterm Birth Among Nulliparous Women. JAMA. 14 mars 2017; 317(10):1047-56.
7. Cobo T, Aldecoa V, Figueras F, Herranz A, Ferrero S, Izquierdo M, et al. Development and validation of a multivariable prediction model of spontaneous preterm delivery and microbial invasion of the amniotic cavity in women with preterm labor. Am J Obstet Gynecol. sept 2020; 223(3):421.el-421.e14.
8. Cobo T, Aldecoa V, Bartha J L, Bugatto F, Paz Carrillo-Badillo M, Comas C, et al. Assessment of an intervention to optimise antenatal management of women admitted with preterm labour and intact membranes using amniocentesis-based predictive risk models: study protocol for a randomised controlled trial (OPTIM-PTL Study). BMJ Open. 28 sept 2021; 11(9):e054711.
9. LeCouter J, Kowalski J, Foster J, Hass P, Zhang Z, Dillard-Telm L, et al. Identification of an angiogenic mitogen selective for endocrine gland endothelium. Nature. 30 août 2001; 412(6850):877-84.
10. Traboulsi W, Brouillet S, Sergent F, Boufettal H, Samouh N, Aboussaouira T, et al. Prokineticins in central and peripheral control of human reproduction. Horm Mol Biol Clin Investig. nov 2015; 24(2):73-81.
11. Brouillet S, Hoffmann P, Feige J-J, Alfaidy N. EG-VEGF: a key endocrine factor in placental development. Trends Endocrinol Metab TEM. oct 2012; 23(10):501-8.
12. Alfaidy N, Hoffmann P, Boufettal H, Samouh N, Aboussaouira T, Benharouga M, et al. The multiple roles of EG-VEGF/PROK1 in normal and pathological placental angiogenesis. BioMed Res Int. 2014; 2014:451906.
13. Gorowiec M R, Catalano R D, Norman J E, Denison F C, Jabbour H N. Prokineticin 1 induces inflammatory response in human myometrium: a potential role in initiating term and preterm parturition. Am J Pathol. déc 2011; 179(6):2709-19.
14. Monnier J, Samson M. Cytokine properties of prokineticins. FEBS J. aout 2008; 275(16):4014-21.
15. Catalano R D, Lannagan TRM, Gorowiec M, Denison F C, Norman J E, Jabbour H N. Prokineticins: novel mediators of inflammatory and contractile pathways at parturition?Mol Hum Reprod. mai 2010; 16(5):311-9.
16. Denison F C, Battersby S, King A E, Szuber M, Jabbour H N. Prokineticin-1: a novel mediator of the inflammatory response in third-trimester human placenta. Endocrinology. juill 2008; 149(7):3470-7.
17. Raia-Barjat T, Prieux C, Gris J-C, Chapelle C, Laporte S, Chauleur C. Angiogenic factors for prediction of preeclampsia and intrauterine growth restriction onset in high-risk women: AngioPred study. J Matern-Fetal Neonatal Med Off J Eur Assoc Perinat Med Fed Asia Ocean Perinat Soc Int Soc Perinat Obstet. janv 2019; 32(2):248-57.
18. Margolis D J, Bilker W, Boston R, Localio R, Berlin J A. Statistical characteristics of area under the receiver operating characteristic curve for a simple prognostic model using traditional and bootstrapped approaches. J Clin Epidemiol. mai 2002; 55(5):518-24.
19. Dunand C, Hoffmann P, Sapin V, Blanchon L, Salomon A, Sergent F, et al. Endocrine gland-derived endothelial growth factor (EG-VEGF) is a potential novel regulator of human parturition. Biol Reprod. sept 2014; 91(3):73.
20. Goldenberg R L, Hauth J C, Andrews W W. Intrauterine infection and preterm delivery. N Engl J Med. 18 mai 2000; 342(20):1500-7.
21. Lannagan TRM, Wilson M R, Denison F, Norman J E, Catalano R D, Jabbour H N. Prokineticin 1 induces a pro-inflammatory response in murine fetal membranes but does not induce preterm delivery. Reprod Camb Engl. dec 2013; 146(6):581-91.
22. Gorowiec M R, Catalano R D, Norman J E, Denison F C, Jabbour H N. Prokineticin 1 induces inflammatory response in human myometrium: a potential role in initiating term and preterm parturition. Am J Pathol. dec 2011; 179(6):2709-19.
23. Catalano R D, Lannagan TRM, Gorowiec M, Denison F C, Norman J E, Jabbour H N. Prokineticins: novel mediators of inflammatory and contractile pathways at parturition?Mol Hum Reprod. mai 2010; 16(5):311-9.
24. Traboulsi W, Sergent F, Boufettal H, Brouillet S, Slim R, Hoffmann P, Benlahfid M, Zhou Q Y, Balboni G, Onnis V, Bolze P A, Salomon A, Sauthier P, Mallet F, Aboussaouira T, Feige J J, Benharouga M, Alfaidy N. Antagonism of EG-VEGF Receptors as Targeted Therapy for Choriocarcinoma Progression In Vitro and In Vivo. Clin Cancer Res. 2017 Nov. 15; 23(22):7130-7140.
25.Cao Y L, Zhang Z F, Wang J, Miao M H, Xu J H, Shen Y P, Chen A M, Du J, Yuan W. Association between polymorphisms of prokineticin receptor (PKR1 rs4627609 and PKR2 rs6053283) and recurrent pregnancy loss. J Zhejiang Univ Sci B. 2016 March; 17(3):218-24.
26 Karaer A, Cigremis Y, Celik E, Urhan Gonullu R. Prokineticin 1 and leukemia inhibitory factor mRNA expression in the endometrium of women with idiopathic recurrent pregnancy loss. Fertil Steril. 2014 October; 102(4):1091-1095.el.
27 Alfaidy N, Hoffmann P, Boufettal H, Samouh N, Aboussaouira T, Benharouga M, Feige J J, Brouillet S. The multiple roles of EG-VEGF/PROK1 in normal and pathological placental angiogenesis. Biomed Res Int. 2014; 2014:451906.

1. An in vitro method for assessing a subject's risk of having or developing spontaneous preterm birth and treating the subject, comprising the steps of i) determining in a blood sample obtained from the subject a level of protein PROK1 marker, ii) determining that the level of protein PROK1 determined in step i) is higher than a reference value and iii) treating the subject determined to have a higher level of protein PROK1 marker with tocolytic or vaginal progesterone medications, a pessary and/or cervical cerclage placement.

2. The in vitro method according to claim 1 wherein the spontaneous preterm birth is detected at an early stage.

3. An in vitro method for monitoring a spontaneous preterm birth in a subject comprising the steps of i) determining a first level of protein PROK1 marker in a blood sample obtained from the subject at a first specific time of the pregnancy, ii) determining a second level of protein PROK1 marker in a blood sample obtained from the subject at a second specific time of the pregnancy, iii) determining that the second level of PROK1 marker is higher than the first level of PROK1 marker and iv) treating the subject with tocolytic or vaginal progesterone medications, a pessary and/or cervical cerclage placement.

4. An in vitro method for monitoring treatment of spontaneous preterm birth in a subject comprising the steps of i) determining a first level of protein PROK1 marker in a first blood sample obtained from the subject before the treatment, ii) determining a second level of protein PROK1 marker in a second blood sample obtained from the subject after the treatment, iii) determining that the second level of PROK1 marker is lower than the first level of PROK1 marker and iv) continuing to treat the subject with the treatment, wherein the treatment is tocolytic or vaginal progesterone medications, a pessary and/or cervical cerclage placement

5. (canceled)

6. (canceled)

7. The method of claim 2, wherein the early stage is at least 20 weeks of pregnancy.
